(19) United States
(12) Patent Application Publication
(10) Pub. No.: US 2015/0297909 A1
(43) Pub. Date: Oct. 22, 2015

(54) APPARATUS FOR ASSISTING WITH THE PHYSIOLOGICAL RECOVERY FROM FATIGUE, STRESS OR INJURY

(71) Applicant: Thomas J. Peashock, Norristown, PA (US)

(72) Inventor: Thomas J. Peashock, Norristown, PA (US)

(21) Appl. No.: 14/690,286

(22) Filed: Apr. 17, 2015

Related U.S. Application Data
(60) Provisional application No. 61/981,056, filed on Apr. 17, 2014.

(51) Int. Cl. A61N 2/00; A61H 39/04; A61H 9/00; A61F 7/02; A61H 1/00; A61N 1/36; A61N 2/02

(52) U.S. Cl. CPC A61N 2/002; A61N 1/36; A61H 39/04; A61N 2/02; A61N 1/36003; A61N 1/36014; A61F 7/02; A61H 1/008; A61H 9/0057

(57) ABSTRACT

The disclosure relates to the process of enhanced recovery from injury or body fatigue due to strenuous activity or strenuous conditions using a contemplated apparatus. Generally the apparatuses disclosed utilize two or more physiological properties and/or body interfaces selected from 1) thermoregulation enhancer via cooling or heating sources; 2) blood flow viscosity enhancer via electromagnetic forces; 3) a circulatory pull by creating a negative pressure or vacuum environment which draws more blood to the treatment area; 4) muscle release apparatus via pressure point applicators and/or electric stimulation devices, which enhance the bodies natural abilities to recover from stress, fatigue or injury; and 5) a fatigue stimulator may be utilized to accelerate the amount of inflammation, stress, and/or fatigue applied to an area of the body via a restrictive cuff which prevent the body from constantly experiencing enhanced recovery techniques and depriving the body of natural adaptation.

### CROSS-REFERENCE TO RELATED PATENT APPLICATIONS

Embodiments of the present invention relate to U.S. Provisional Application Ser. No. 61/981,056, Filed Apr. 17, 2014, entitled “APPARATUS FOR ASSISTING WITH THE PHYSIOLOGICAL RECOVERY FROM FATIGUE, STRESS OR INJURY”, the contents of which are incorporated by reference herein and which is a basis for a claim of priority.

### BACKGROUND OF THE INVENTION

1. Field of the Invention

The invention generally relates to a physiology assistance device capable of assisting a user recover from a physiological stress, fatigue, inflammation or injury condition, and more specifically to an apparatus that assists with thermoregulation, circulatory enhancement device, and muscle oxygenation and release techniques for injured, seized, fatigued, stressed or overheated muscles.

2. Background Information

People are constantly faced with physical challenges, either voluntarily when working in physical occupations, working out to attain a higher level of fitness, or in athletic activities, or involuntarily during strenuous exertion of daily life. When the body and primarily muscles reach a point of fatigue or stress it becomes necessary for the user to stop the activity and rest to allow the bodies natural physiological recovery system to guide the body back to stasis.

Although, there are many sports medicine related devices on the market which attempt to assist the physiological recovery process, covering various aspects of physiological needs, none of the devices reviewed comprised of a single unit capable of performing various physiological enhancements at the same time which may assist the user's recovery and adapt to a condition of fatigue, stress and/or injury. Additionally, the recovery device may comprise a fatigue stimulator to rapidly expose users to a condition of inflammation, stress, and/or fatigue. The fatigue stimulator provides the user with an option to counteract the recovery techniques present in this invention which may be depriving the body from its natural adaptation.

### SUMMARY OF THE INVENTION

The embodiments of the present invention generally relate to the process of enhanced recovery from injury or body fatigue due to strenuous activity or strenuous conditions. More specifically the embodiments of the present invention utilize two or more physiological properties and or body interfaces selected from 1) thermoregulation enhancer via the use of cooling or heating sources; 2) blood flow viscosity enhancer via the use of electromagnetic forces; 3) a circulatory pull by creating a negative pressure or vacuum environment which draws more blood to the treatment area and/or a 4) muscle release apparatus via the use of pressure point applicators and/or electric stimulation devices, which work together to enhance the bodies natural abilities to recover from stress, fatigue or injury. To counteract these recovery enhancements that prevent the body from experiencing and overcoming inflammation, stress, and fatigue, a 5) fatigue stimulator is present via the use of a restrictive cuff to increase metabolic stress and adaptation by restricting the flow of blood to an applied area and a 6) heat source to increase inflammation, heat stress and heat fatigue.

An embodiment of the present invention includes a device that reduces blood viscosity and increases blood flow, while changing the blood's temperature and possibly restricting blood flow comprising of a negative pressure environment, negative pressure generator, heat exchange surface, appendage, magnetic field generator, pressure point presser, and fatigue stimulators.

An exemplary embodiment of the present invention comprises a vacuum sealed, shrink wrapped rubber sleeve that has a suction valve squeeze pump attached to it to create, and hold a negative pressure environment while enclosing an appendage such as the hand or foot and a portion of the arm or leg. The invention may also be comprised of a gel pack, which acts as the heat exchange surface. The gel pack may be secured to the hand or foot via a magnetic strap, which has magnets attached to the part of the strap to create a magnetic field on the hand/wrist area or foot/ankle area. A fatigue stimulator may also be comprised of a stretchable Velcro strap capable of restricting the blood flow of an appendage. While a heat pack can provide hours of heat cheaply and conveniently acting as another possible long lasting fatigue stimulator.

Additional embodiments may include a sleeve cast cover which may provide a partial or fully sealed negative pressure environment. An embodied apparatus may also comprise of a gel pack, and strap that secures 1.3 tesla Neodymium magnets to the hand/wrist area while also securing the gel pack to the palm of the hand. A negative pressure environment may be made by making a sleeve comprising rubber, neoprene, or any other air-tight flexible sleeve material in conjunction with a negative pressure generator. A negative pressure generator may be made by inserting a suction valve into the sleeve, and attaching it to a suction squeeze pump with a hole at the top to suck the air out of the enclosed sleeve. An elastic and/or hook and loop fastening system such as Velcro may be used for a stretching strap which may act as the seal at the end of the sleeve, while also acting as a fatigue stimulator if stretched tight enough to restrict blood flow.

Further additional embodiments may include a muscle release facilitator which may be in the form of a pressure point applicator which may compress against a seized or tight muscle mass and cause a reactionary muscle firing response and a gradual release of the tightened muscle mass when the triggering pressure point applicator is removed. Another muscle release facilitator contemplated may be in the form of an electrical stimulation (e-stim) device which applies electric pulses to muscles which forces them to fire and contract, when the device is removed the exhausted muscles release into a more relaxed state.

### BRIEF DESCRIPTION OF THE DRAWINGS

The principles of the present invention will be apparent with reference to the following drawings, in which like reference numerals denote like components:

FIG. 1. Is an exterior view of an embodied device designed for use with an arm and hand extremity.

FIG. 2. Is an exterior view of another embodied device designed for use with an arm and hand extremity, but without a negative pressure embodiment.

FIG. 3. Is an exterior view of yet another embodied device designed for use with an arm and hand extremity.

FIG. 4. Is an exterior view of yet still another embodied device designed for use with an arm and hand extremity.

FIG. 5. Is an exterior view of an additional embodied device designed for use with an arm and hand extremity.

FIG. 6. Is an exterior view of yet another embodied device designed for use with a hand extremity.

FIG. 7. Is an exterior view of another alternative embodied device while in use with the hand and wrist of a user but without a negative pressure embodiment, and also contains a circulating fluid apparatus that is fastened to the shoulders and back of a user.

FIG. 8. Is an exterior view of an embodied device designed for use with the foot and lower and upper leg.

FIG. 9. Is an exterior view of another embodied device designed for use with an arm and hand extremity.

### DETAILED DESCRIPTION OF THE INVENTION

The embodiments of the present invention generally relate to the process of enhanced recovery from injury or body fatigue due to strenuous activity or strenuous conditions. More specifically the embodiments of the present invention utilize two or more physiological properties and or body interfaces selected from 1) thermoregulation enhancer via the use of cooling or heating sources; 2) blood flow viscosity enhancer via the use of electromagnetic forces; 3) a circulatory pull by creating a negative pressure or vacuum environment which draws more blood to the treatment area and/or a 4) muscle release apparatus via the use of pressure point applicators and/or electric stimulation devices, which work together to enhance the bodies natural abilities to recover from stress, fatigue or injury. To counteract the recovery benefits that make it easier on the body, and prevent the body from overcoming its natural response to inflammation, stress, and/or fatigue; a fatigue stimulator may be present to rapidly apply inflammation, stress and/or fatigue to the body's muscles via a cuff that restricts the flow of blood and oxygen to the muscles. While the use of a heat pack can provide hours of heat and can also act as fatigue stimulator.

Embodiments may include all of the above physiological recovery enhancers or any combination of at least two enhancers such as, but not limited by, a) thermoregulation with blood flow viscosity, b) thermoregulation with circulatory pull enhancers, c) thermoregulation with muscle release enhancers, d) blood flow viscosity with circulatory pull enhancers; e) thermoregulation with blood flow viscosity and muscle release enhancers; and f) blood flow viscosity and muscle release enhancers with circulatory pull. All of the physiological recovery enhancers may be used in combination with the fatigue stimulator, which may also be used independently on its own.

In embodiments wherein the technique to reduce blood viscosity is combined with the technique to cool blood, one may experience an enhanced efficiency in cooling the body, and in muscular performance. Both techniques bring unique traits that influence the cooling down of the body, while also enhancing muscular performance. Being able to reduce blood viscosity may aid the cooling process of the body, as the increased blood flow helps circulate the cooled blood throughout the body, and allows it to complete its flow cycle in a reduced amount of time. The increased blood flow cycle may also help to enhance muscular performance by reducing metabolic waste build up, as the enhanced blood flow increases the efficiency in the metabolic waste removal during and after exercise. Another benefit of reduced blood viscosity on muscle performance is that it increases the flow of oxygen-rich blood to working muscle. The increased volume in blood oxygen supply levels may help the body's muscles perform at enhanced levels, as the muscles are exposed to an increased supply of oxygen that is needed to function. Meanwhile, cooling the blood may help muscular improvement by removing heat from the muscles, which allows them to operate at enhanced levels, as cooling the blood in turn cools the muscles and combats muscle fatigue and heat stress.

Still further embodiments assist with different ways to reduce blood viscosity/increase blood flow and may include, but are not limited to the ways or examples described herein. Medically, it is possible to reduce blood viscosity by blood transfusions or by orally taking Aspirin, Coumadin (Warfarin), and other manufactured drugs that are known to reduce blood viscosity. Meanwhile, naturally occurring blood viscosity reducing substances can be found in: Curry powder, Cayenne pepper, Ginger, Paprika, Thyme, Cinnamon, Dill, Oregano, Turmeric, Licorice, Peppermint, Raisins, Prunes, Cherries, Cranberries, Blueberries, Grapes, Strawberries, Tangerines, Oranges, Chewing gum, Honey, Peppermints, Vinegar, Wine, and Cider. Compression garments and gradient compression garments can manipulate blood flow. A natural technique that could stimulate blood flow occurs from active movement or through deep breathing exercises, stretching or muscular massages, and other known pain relieving techniques. Scientifically, an object capable of creating a magnetic force or electromagnetic force in the same direction as a mammal's blood flow could reduce blood viscosity and thus increase blood flow. Generally, the magnetic force used to reduce blood viscosity is between 1-3 tesla, however it may be possible to facilitate increased blood flow at lower tesla. Applying force to pressure points or receiving acupuncture/acupressure treatment is another way to stimulate blood flow. Applying heat to an area via a hot pack or heating pad will also stimulate blood flow to a targeted area. Hot and cold showers, alternating between hot and cold surfaces, or applying hot/cold creams can also help to stimulate blood flow. Lastly, gene doping/manipulation or hormone replacement therapy could provide an individual with advanced scientific ways where one may experience enhanced abilities to reduce their blood viscosity.

Additional embodiments, which provide different ways to create a negative pressure environment inside an enclosure, may include but are not limited to the ways or examples described herein. Air pumps, tube pumps, vacuums, fans, and filtration/circulation systems may all be used to create negative pressure environments inside an enclosure. Also, a vacuum may be created by inhaling into an opening in the air tight enclosed material while breathing, sucking out all of the air, and creating a negative vacuum pull inside the enclosure. While the user inhales, their hand could be used to create and keep the air tight seal between their mouth and the sealed enclosure.

Additional embodiments, which create a heat exchange surface may include, but are not limited to the ways or examples described herein. Heated or cooled water can be drawn from an isolated supply chamber, which is meant to cool or heat the water, and then distribute the water from the main supply chamber via a tubed filtration/circulation system. The tubed filtration/circulation system can then be run through, and in contact with a surface area that conducts heat well, such as metal, or aluminum. Other examples of heat exchange surfaces may include other metal surface areas, gel packs, chemical substances like icy/hot creams or hot packs, electrically cooled or heated surface areas, cooling/heating blankets, and other substances similar to rice, beans and/or rock pebbles that can hold heat.

Further additional embodiments including alternative ways to create a vacuum sealed enclosure may include but are not limited to the ways or examples described herein. A vacuum seal may be created by using any housing structure that houses a foot/leg or a hand/arm inside of it. Variations of housing may allow one to create a hard seal, where air leakage can not occur. Or a soft seal, which creates a negative pressure environment, but the material caves inward, and/or the sealed material very slowly leaked air; a soda bottle would be an example of how plastic can cave inward under negative pressure. Other ways to create a vacuum sealed enclosure may stem from using a balloon, bag, or sleeve. Sleeves can vary in length, seal strength, and shape. The length of a sleeve can vary to cover the user's hand and wrist, or to cover their hand and arm, which could extend past the elbow. Likewise, the length of a sleeve can vary to cover the user's foot and ankle, or to cover from the foot up to, or past the knee. The seal strength on the sleeve can also vary between being able to hold negative pressure inside of the sleeve with minimal to no air leakage to not being able to hold negative pressure inside the sleeve. If the sleeve is unable to hold a negative pressure environment, then the negative pressure environment could be created from the vacuum itself. In instances where the sleeve has an imperfect seal, a vacuum or tube pump allows the sleeve to maintain a negative pressure environment at the very end of the sleeve, where the air is being withdrawn from the enclosed sleeve. The shape of the imperfect sealed sleeve changes between temporarily conforming to the appendages like shrink wrap while the vacuum is in affect, to residing in its natural state when no vacuum is present or provided. In instances where the sleeve has a perfect seal, there is minimal to no air leakage inside the sleeve, as the material creates an airtight vacuum seal, and conforms to the user's appendages. The shape of the perfectly sealed sleeve in use is like a shrink wrap as the sleeve conforms to the arms/legs and their appendages, and is able to maintain the shrink wrap appearance because no air can escape.

Additional embodiments may include a muscle release facilitator which may be in the form of a pressure point applicator which compresses against a seized or tight muscle mass and causes a reactionary muscle firing response and a gradual release of the tightened muscle mass when the triggering pressure point applicator is removed. Another muscle release facilitator contemplated may be in the form of an electrical stimulation (e-stim) device which applies electric pulses to muscles which forces them to fire and contract, when the device is removed the exhausted muscles release into a more relaxed state.

Additional embodiments include a combination of the devices and physiological or pharmacological devices that can impact core body temperature, and increase one's blood flow and/or simultaneously affect one's blood viscosity, create a negative pressure environment in an appendage enclosure, and/or create a heat exchange surface. If one were to remove the process of blood viscosity, then one would still be able to use thermoregulation to impact the body's core temperature. Likewise, if one were to remove the thermoregulation process, then one would still be able to experience the benefits of reduced blood viscosity. Another example of an embodied device that impacts core body temperature and blood viscosity is made using a vacuum sealed sleeve enclosure with an enlarged elongated end piece that expands into a balloon shape when filled with air. Attached to the sleeve may be a sphygmomanometer pump, which can pump air into the end of the sleeve and fill the large balloon shaped part of it with air that can then be slowly released to create an extended vacuum.

Additional embodiments apply an embodied device to influence body temperature, and blood viscosity by comprising the following steps. First, one's hand is placed into an enclosed sleeve, and the hands take hold of a magnetic aluminum or copper can, which is setup to a tubed circulation system pumping 40-80 degree Fahrenheit water/liquid through the can. To start the process, the user squeezes air into the enclosed sleeve, filling the balloon part of the sleeve with air. The beginning, opening, or top part of the sleeve that assists with thermoregulation may be tied down with a string to create a fatigue stimulator and to secure the vacuum tight seal enclosure. Once filled with air, the user loosens the release valve on the sphygmomanometer pump to create a slow and steady vacuum from the sleeve. The magnets in the can help increase the blood flow/circulation; while a steady vacuum present from the air exiting the ballooned enclosure sucks the blood towards the palms/soles of the hands/feet, where it is then cooled by the magnetic aluminum can before the cooled blood is sent back through the veins, past the muscles, and to the heart. This process may be used before, during, or after the body has performed work by a user seeking an all natural blood circulation/muscle performance enhancer. Additionally, a fatigue stimulator may be utilized by securing the string around the arms/legs and restricting approximately 50-70% of the blood flow.

Further embodiments may be alternatively designed through common sporting gloves such as boxing gloves, hockey gloves, baseball batting/fielding gloves, and MMA style gloves (plus other types of fighting gloves). And also alternatively designed through boots such as ski boots, ice hockey or roller skates, water sport boots, sports cleats (football, baseball, soccer, golf, rugby), and sports shoes (running, basketball, track and field, tennis, and golf shoes).

Additional embodiments cover uses wherein there is a need to reduce blood viscosity in the prevention and/or treatment of blood clots or any other blood circulation condition. Also, this device may be used in sports medicine to treat injuries, and to relieve pain because it can improve efficiency in blood flow and fluid exchange that helps reduce pain and inflammation caused by injured tissue, which stimulates the healing process. Similarly, the practice of magnetic wave therapy may be seen as an alternate use. The applications usefulness may also be extended to physical therapy, and to muscle relaxation techniques such as body massages, as the increased blood flow aids in the massages overall effectiveness.

Another embodiment applies to use under the thermoregulation field in the medical industry is that it could be used to prevent/combat hypothermia. Another use could be to combat dangerous body heat levels caused by fevers, natural ailments, and chemical reactions/allergies. Lastly, any field of work or activity where one is exposed to prolonged periods in hot environments could be considered a possible alternative user, as this device could aid them in preventing heat related illnesses.

Stimulating pressure points can activate the body's natural healing and self-curative abilities as it can enhance blood circulation, circulate the body's vital energy (Qi), release muscular tension, reduce pain, and enable the body to relax deeply. To receive maximum benefit when pressing points, pressure should be applied for at least half a minute, but ideally for as long as one to three minutes or longer if desired. A blunt object should be used to provide gradual, steady, 90 degree penetrating pressure to the surface of the skin.

Pressure points manipulators may be incorporated into the device through the following means:

a) Secured under the top material of the end appendage device, solid blunt objects are aligned along selected pressure points to apply vertical pressure to the targeted pressure points found on the hands/arm and feet/legs. The pressure points found in the hands/arms and feet/legs comprises the finger webs, thumb webs, fingers, inside wrist, outside wrist, forearm, outside elbow (Ubiar Nerve), inside elbow (Radial Nerve), triceps, foot instep (2^{nd}/3^{rd} Metatarsal), back of knee, inside of knee, inside of ankle, back of ankle (Achilles Tendon), among others.

b) Secured under the top material of the blanket device, solid blunt objects are aligned along selected pressure points to apply vertical pressure to the targeted pressure points found on the back/chest area, while the underneath layered material may be small egg carton shells or needles used to apply pressure and relieve tension/stress on the underlying muscles found in the chest/back surface area.

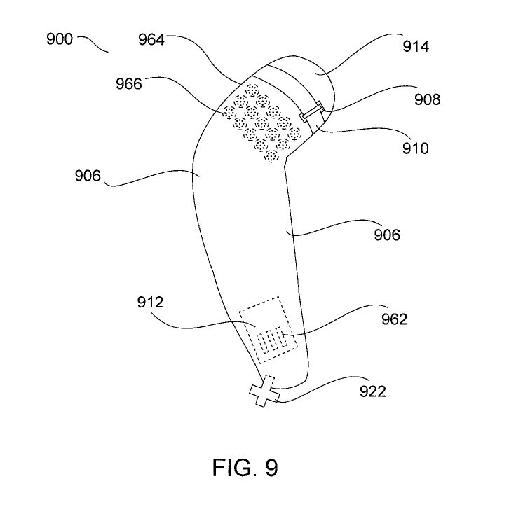

The embodied pressure point system demonstrated in FIG. 9 is an example of the point system in use on the arm. Another embodied application would be an upper body application which encompasses the upper arms, shoulders, chest, and upper back. This may be accomplished with a vest like apparatus with sleeves attached to it to cover the top of the arms. Fastened to the inside of the vest and arm sleeves may be round sharp plastic points that may be applied to one's skin to trigger release points in those applied areas and loosen the muscles before a workout. The point system works by pressing the points against the body in various ways including but not limited to the ways and examples described herein. When in use the user lays against the ground or against an object to apply one's body weight to create pressure, the user may also use one's body parts to create pressure such as by using their hands, and may also place weight on top of the point system to create pressure. Another embodied system may include a weighted vest apparatus where sand, water, or other substances are used to create pressure on top of the plastic pressure points found underneath the vest-like apparatus. Another alternative includes a system similar to that found in FIG. 9, but with an inflatable tube, wherein the tube is inflated via a pump attached to a hose. The inflatable tube lines the inside of the vest-like apparatus and when inflated applies pressure to the plastic points, which then applies pressure against the skin of the user. Another embodied system involves overlapping the material, where the user tightly wraps and fastens the material around their arms, shoulders, chest, and back.

The benefit of using a negative pressure sleeve or creating a negative pressure environment with a point pressing system would be the increased force that the negative pressure sleeve or environment applies to the pressure points inside the sealed enclosure. Pressing points would increase the force applied to all of the pressure points in the sealed enclosure. The blunt pressure points could be secured under the sealed enclosure and provide a continuous and steady pressure to ones appendage due to the negative pressure environment, which creates an enhanced muscle release facilitator system. If no negative pressure is present, then the point pressing system would need to be tightly wrapped around an appendage to act as a muscle release facilitator.

Some embodiments of the invention may work through a combination of blood manipulating techniques. Pressing pressure points may enhance blood circulation and relieve muscular tension allowing for an increased supply of oxygen to reach the muscles and targeted areas.

Additional embodiments used to restrict blood flow may include, but are not limited to the ways or examples described herein. Typically a blood flow restricting cuff or tourniquet may be used, but any material/object that can impede the flow of blood to another area of the body could be used. Cuffs to restrict blood flow may be comprised of belts, tie downs, inflatable materials that surround body parts, string or Velcro material wrapped tightly around body parts, hook and pull Velcro straps, fastening an additional layer of stretched out tighter woven material to the underlying material, and/or physically by using one's hands or other body parts to impede the natural flow of blood to another area of the body. The cuff may be used in connection with the other recovery benefits present in the device to enhance, assist, or counteract their benefits. For example, a cuff would enhance the pooled blood's exposure to a hot/cold exchange surface in the palms/soles, which would also be aided by the negative pressure generator, whereas the restriction of blood flow at the tops of one's arms/legs will help pool blood in the hands/feet by slowing the blood flow in and out of those areas. The increased contact duration between the palm's/sole's blood and the hot/cold exchange surface may thus assist in the overall effectiveness of the recovery system's ability to directly cool the muscles. Additionally, the increased blood flow cycle brought forth from one's exposure to magnets will continue for the overall body even with an isolated restrictive cuff on the arms/legs. Meanwhile, the pressure point system may counteract the built up inflammation caused by the restrictive cuff when the pressure points are applied to the targeted area by flushing in oxygen to the targeted area and accelerating or kick starting the recovery process. As a whole, the recovery system/device aids the performance of the body during physical activity, while the restrictive cuff may act as a fatigue stimulator to expose the body to increased levels of inflammation, stress, and/or fatigue.

Additional embodiments to replicate an inflammation, stress, or fatigue stimulator may include, but are not limited to the following ways or examples. Artificial or extreme environments where the body is exposed to heat, cold or humid environments. Artificial or extreme environments where the body is exposed to different elevation levels. Increased stress may be placed on the body through weighted garments such as strap on ankle weights, weighted belts, or a weighted vest. A restrictive cuff would act as a fatigue stimulator by depriving the targeted area oxygen and blood flow. Sleep deprivation could be another way to stimulate fatigue in the body. Fatigue can be stimulated by constant muscle use, where the muscles are worked until failure. The body can be exposed to inflammation through any actions that results in pain, redness, heat, swelling, or loss of mobility. The body can take in inflammation orally by digesting foods high in trans or saturated fat. Lastly, the body can be exposed to elevated levels of inflammation by eating too much, living an inactive lifestyle, stressing out, and not receiving enough sleep.

Blood flow restriction training is generally considered safe in healthy individuals, however one of the main risks associated with blood flow restriction training is the risk of embolism, which is the formation of blood clots. Some embodiments of the present invention comprise a tight sleeve that is similar to a compression sleeve, which may aid blood flow and help prevent the formation of blood clots. When the tight sleeve does not hold a negative pressure environment completely, the negative pressure generator, squeeze pump, helps create a gradient compression at the end of the sleeve/arm. In this environment, one's hands experience the greatest amount of pressure when the squeeze pump is pumped, and that pressure gradually dissipates over a 10 to 30 second timeframe, which may last longer depending on how well the material holds a negative pressure environment and how breathable the underlying material is. If the underlying material of the sleeve is made of nylon or a non-rubber material, then the sleeve will not hold a negative pressure environment without a constant pump. In this environment, maximum pressure is applied to the hand area where blood is drawn and pooled in the hands due to the vacuum found at the end of the sleeve. The pulling of blood to the hand area and then the gradual loss of air in the sleeve helps keep the blood flowing freely through the hands and arm, as the pressure is constantly changing. If the underlying sleeve material is more airtight such as when a sleeve is made from rubber, then it will hold a negative pressure environment by itself. The compression found in this type of sleeve would be constant, while the pressure is equally dispersed throughout the sleeve.

Magnets may also assist in the prevention of blood clots, as the iron found in the red blood cells may be polarized with magnetic fields that cause the red blood cells to link together in short chains, streamlining the flow of blood through the veins and decreasing the blood's viscosity. An alternative make using magnets to draw or pool blood could occur in the form of a magnetic wrap. Where magnets could be sewn in or placed under the fabric with the goal of surrounding the top of the arm's bicep, tricep or forearm muscles, which may help recovery through increased blood flow and oxygen.

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

### Example 1

### Hand/Arm Device

An exemplary embodiment of the invention includes a hand/arm device intended for use to enhance the muscular performance and blood flow of a user through the manipulation of the blood's temperature and viscosity through natural scientific procedures. The apparatus may include a heat exchange surface comprising a gel pack between 40-80 degrees Fahrenheit prepared by placing the gel pack into a cold storage unit or running it under a cold water faucet. One may then put on the magnetic strap around the top of the hand, and secure the prepared cold gel pack into the palms and around the strap. Then, one may insert their hand/arm holding the cold gel pack/magnetic strap into the vacuum sealed, shrink wrap rubber sleeve, and use the squeeze pump to completely remove the air from the sleeve, so that the sleeve maintains a negative pressure environment. After these steps, one may experience the benefits of muscular enhancement, and increased blood circulation after waiting a time period that could be comprised of 30 seconds, and up to 5 minutes, or longer if desired. The embodied apparatus and methods may be used before, during, or after the body has performed work, in order to enhance one's muscular strength and/or endurance through the duration of one's work interval.

Additional embodiments of the present invention may work through a combination of blood manipulating techniques. The first to occur is when the magnetic strap is placed on the back of the hand or wrist area. Magnets between 1-3 tesla may be secured on a strap contacting the knuckles, top/bottom of hand, or wrist area, which would facilitate blood flow within 1 minutes time, as the magnetic fields may be able to impact the blood flow through the surface veins residing along the skins surface. The magnetic fields impact the blood because the blood carries red blood cells, which carry the iron-based protein hemoglobin. When the magnet's magnetic field is applied parallel to the direction of blood flow, the magnetic field polarizes the red blood cells causing them to link together in short chains, streamlining the movement with the direction of blood flow. This in turn sends the blood back to the heart faster, which allows for an increase in blood circulation/distribution throughout the body.

A further embodiment occurs after body has begun to experience an increase in blood/oxygen circulation. At this time, the hand/arm holding the cold gel pack/magnets is placed into the vacuum sealed, shrink wrap sleeve, and the squeeze pump is used to completely remove the air from the sleeve. This process creates a negative pressure environment inside the vacuum sealed enclosure. The negative pressure environment causes the blood to be pulled towards the palm's surface, where the capillaries in the palm help remove heat from body. The palm is used specifically because it is one of the body's main radiators for heat dispersion. While the blood is being pulled towards the palms the sleeve is also tight enough that it may alter the flow of blood out of the hand. The pooled blood in the palms is exposed to the cold gel pack, which is cooling the pooled blood in the palms before the blood is sent back through the veins, past the muscles, to the heart, and then disbursed throughout the body. Cooling the blood, in turn helps to cool the entire body, which may help prevent muscles from being over heated. Removing heat from the muscles may allow them to exceed their natural capacity, as the muscles don't fatigue as quickly as normal, and thus are able to do more work. The arm muscles receive the greatest benefit, as the forearm, bicep, and tricep muscles experience the coldest blood flow which directly combats heat fatigue in those muscles and promotes endurance during arm exercises. It may also be possible that palm cooling overrides pain sensors between the brain and the muscles, which alters the muscle to brain connection during exercise and may allow for an increased volume of work. Meanwhile, the shrink wrapped sleeve aids in supplying the arm's muscles with the coldest blood flow, as it squeezes the veins and lessens the distance that the blood must travel between the palms and the arm's muscles.

An alternative make of a cold pack could be in the form of a multi sided gel pack that fits around the palm and back of the hand, which allows for an increased rate of thermoregulation compared to a single sided gel pack, which is limited to only covering the surface area of the palm. It should be understood that the multi sided gel pack may replace the single sided gel pack on any of the devices that used a single sided gel pack. A thermoregulation component that cools both sides of the hand sends cold blood back up the arm through all the veins found in the arm. The deep veins found in the arm may have the greatest impact in cooling the muscles when carrying the cooled blood and may be the most useful veins in combating heat fatigue in the arm's muscles. The cooling process would be enhanced with a negative pressure sleeve 100 that slows the blood flow out of the hand area allowing for an increased contact exposure time with the multi sided gel pack. The actual flow of the cold blood from the hands through the arm may occur as follows: The blood flow in the back of the hand drains into the dorsal digital veins, which then drains to the dorsal metacarpal veins, which drains to the dorsal venous network, which then drains into the cephalic vein & basilic vein, which drains to the median cubital vein and finally into the axillary vein, which is where the blood leaves the arm and makes its way towards the heart. Meanwhile, the blood flow in the palm of the hand drains into the common palmar digital veins, which drains to the intercapitular veins and the superficial palmar venous arch, which drains to the ulnar vein, which drains to the brachial veins and finally into the axillary vein, which is where the blood leaves the arm and makes its way towards the heart. Or if the blood flows to the intercapitular veins, it then flows to the median antebrachial vein, which flows to the basilic vein and median cubital vein, which finally drains to the axillary vein and leaves the arm.

Cold gel packs are cheap and convenient, and are easily transported with a user for use as a thermal regulator as an excellent source of cold that can last for hours when stored before use inside a cooler or other cooling agent and then can be applied for 5-20 minutes between 40-80 degrees Fahrenheit. However, heating those same gel packs, while possible, does not provide an extended use of heat beyond the initial heating phase that results in roughly 2 minutes of heat between 100-140 degrees Fahrenheit, as the gel packs do not retain heat well. As a result, a better solution to act as a hot thermal regulator would be chemical heat. Where a chemical mixture of iron powder, water, salt, activated charcoal and vermiculite can lead to hours upon hours of heat between 100-140 degrees Fahrenheit. A popular product that uses this chemical solution for heat is HotHands, which is one of the most convenient and affordable ways to apply heat.

A still further embodiment includes the embodied device paired or synced to a phone app, mobile device, or other device capable of displaying, monitoring or tracking your results. Through the use of motion sensors or global position system (GPS) technology, which could use an add on body clip capable of calorie tracking, strength tracking, or monitoring other applicable health or fitness tasks while wearing the embodied device. Tracking performance data may allow the device to include buddy system apps capable of providing competition to other users when using the invention, and also allow for live recommendation tips to enhance the end user's experience when using the invention. Meanwhile, when resting between sets, the user's mobile device may be synced with a finger sensor technology capable of gathering a user's heart rate and heart rate variability. Collecting this data could provide a user with up to second data on heart rate recovery, which could signal to the user that they are fully recovered from a previous bout of exercise and can start the next set of exercises. This could be accomplished by using a finger sensor with an infrared light that emits light from one end of the finger sensor to the other end of the finger sensor where the sensor measures the changes in the amount of light received as blood is constantly pumped around the body and the blood density changes in the finger with every heart beat, which is the information the finger sensor transmits wirelessly to the a user's phone. A wire could also be run through the shrink wrapped recovery sleeve and plugged into a phone, which would connect the phone, finger sensor and user. An algorithm could take the collected data and display an accurate heart rate variability measurement, as if it had been derived from a high end electrocardiograph, to the user of the embodied device. While a finger sensor is capable of providing this information, a wrist strap or watch my also be able to collect heart rate recovery data and display it to the user via wireless transmission and collected through infrared, light-emitting diode (LED) or photo sensors. While this data may be obtained on the recovery arm, it should also be understood that it could also be obtained from the non-recovery arm.

Attached to the negative pressure athletic sleeve may also be infrared sensors or other sensors capable of monitoring and displaying data onto a hand held device (i.e. phone) of the body's temperature or the temperature of the arm muscle's, such as the forearm, biceps, or triceps. This information may be used by the end user during their rest intervals to dictate how long they need to use the invention to combat heat fatigue, which in turn will help provide optimal results when using the invention. Embodied sensors may also be capable of monitoring the cold source, or cold gel pack that is in contact with the hand to help optimize use. The process of collecting and monitoring the temperature of the cold gel pack will allow for end user notifications as to when the cold gel pack should be replaced or removed.

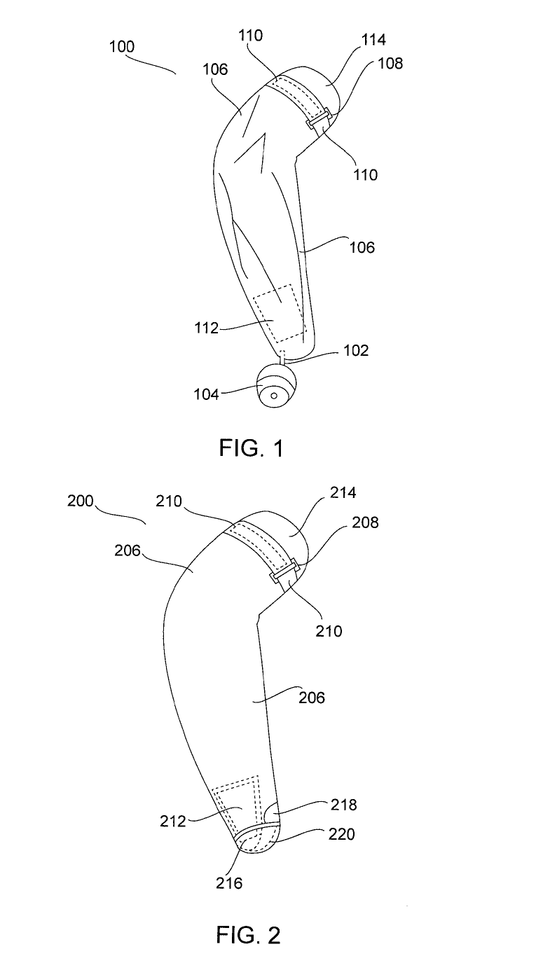

FIGS. 1-9 exemplary but non-limited recovery devices which may be worn before, during or after exercise to enhance physiological recovery, perceptual recovery and recovery from delayed onset muscle soreness (DOMS). It should be understood that physiological recovery relates to physical changes in the body that result in improved recovery or performance, which comprise an increase in muscle size, strength or flexibility; while it may also comprise chemical changes in the body such as in testosterone, human growth hormone (HGH), lactate levels and cortisol to name a few. It should also be understood that perceptual recovery deals with a user's perception of utilizing a recovery technique to improve their recovery from exercise. Generally, perceptual recovery maybe thought of as the placebo effect or the belief effect, and may typically occur after a positive benefit is associated with the improvement of recovery or performance. If perceptual recovery is enhanced, a user may have an increased drive to finish a race or set that they might not of completed without the perceptual aid. Likewise, if perceptual recovery is enhanced, a user may experience less DOMS if they actually believe that the recovery steps they are taking help make a difference. It should also be understood that recovery from DOMS is the recovery of post-workout muscle soreness that typically occurs 24-72 hours post-exercise. Enhancing the physiological and perceptual recovery of a user may also enhance a user's recovery from DOMS.

Yet further additional embodiments may be used to widen blood vessels or increase vasodilation that may help increase the transfer of heat from the body which may include, but are not limited to the ways or examples described herein. Increased vasodilation may occur through eating more nitrate rich foods or increasing nitrate oxide intake into the body. It may also be increased through exercise or physical activity. Another way to increase vasodilation is through carbon dioxide (CO₂) or increased carbon dioxide (CO₂) exposure. Yet another way to increase vasodilation is through the exposure of heat which will help widen the blood vessels. Also, vasodilation may be enhance via exposure to a negative pressure environment. The present invention may increase vasodilation via a negative pressure environment which may enhance recovery from heat stress, heat fatigue or inflammation. The application of heat before, during or after exercise may allow for increased vasodilation which may enhance heat transfer if a cold source is applied immediately after the heat source. Likewise, the use of blood flow restriction may be applied before, during or after exercise which may allow for an increase in vasodilation resulting in an increase in heat transfer immediately after application.

The following physiological recovery enhancers may be present. First, the use of thermoregulation enhancer capable of altering a user's body temperature which may decrease inflammation and improve recovery time. The use of a thermoregulation enhancer may also help improve heart rate recovery as cooling the body could lower heat stress which may result in improved heart recovery. Second is the use of blood flow enhancers that may improve blood flow via the use of magnets or through negative pressure compression. The use of magnets may streamline the iron in the blood to allow for increased blood flow, or allow for an increased amount of blood to pool in one area where magnets are placed which may allow for an increased delivery of oxygen to fatigued muscles to improve recovery. The use of a negative pressure shrink wrap or compression sleeve comprising a negative pressure environment, sleeve covering and user's appendage may improve blood flow by squeezing an appendage and the surface veins which shortens and decreases the distance blood needs to travel which may improve blood flow and delivery of oxygen to improve recovery and repair fatigued, stressed and inflamed muscles. Third is the use of a negative pressure or vacuum environment to pull blood to the surface of the treatment area and increase the rate of heat exchange at the skins surface through the widening of blood vessels which is known as vasodilation. Fourth is the use of a muscle release apparatus that may alter blood flow and aid in the stretching of muscles through self-myofascial release. Last is the use of electric stimulation where the firing and contraction of muscles stimulates blood flow which may improve recovery through increased exposure to oxygen.

The following perceptual recovery enhancers may also be present which may help increase performance and recovery. First, a cold thermoregulation enhancer that feels good when used in hot conditions and may be perceived to help improve recovery. It may also lower a user's perception of pain which may lead to enhanced performance or recovery. While a hot thermoregulation enhancer may feel soothing and may be perceived to help improve recovery or performance. Second, blood flow enhancers may also be associated with improved recovery. For example, magnets may be associate with an increase in blood flow which may then lead to a greater perceived recovery in an individual. Third is the use of a negative pressure or vacuum environment with a sleeve covering that feels good when in use and may be associated with an increase in perceived recovery. Fourth is the use of a muscle release apparatus that feels good when applied to the skin and may also be associated with an increased perceived recovery. Lastly is the use of electric stimulation which may be associated with an increased in perceived recovery because a user feels the muscles squeeze when in use and may think it helps.

Physiological and perceptual recovery techniques help with the recovery from delayed onset muscle soreness (DOMS). The physiological recovery techniques may create changes in the body that lead to less DOMS by reducing stress, fatigue or inflammation. While users of perceptual recovery techniques may use a positive event or an association with something that feels good as belief that it enhances their recovery or performance which then leads to enhance recovery or performance.

While the use of recovery techniques could help increase adaptation through recovery, it could also hinder adaptation if a user constantly makes things easier on their body. As a result, the following counter measures or super-compensation strategies may be used to enhance stress, fatigue and inflammation which could lead to increased adaptation. First is the use of a restriction cuff that slows the flow of blood into a limb while it also pools the blood behind the cuff. Second is the use of a heat source that may increase heat stress, heat fatigue and inflammation.

While this invention generally deals with ways to recover from stress, fatigue and inflammation. It's also the goal of the present invention to measure and report the rate of recovery or the recovery stats to the end user. Heart rate recovery as measured by heart rate variability (HRV) or heart rate could be measured with a HRV sensor inside the sleeve or on the opposite appendage. While temperature sensors could be placed inside the recovery sleeve to measure skin and muscle temperature. Lastly, the use of perspiration collectors could help obtain, process or distribute data to the user comprising lactate levels, lactate clearance, blood oxygen saturation percentages, cortisol, creatine kinase and dehydration testing among many others. As other means may exist to measure recovery of stress, fatigue or inflammation not currently disclosed.

While the invention may be used for other means, some common uses with weight training may comprise 1) use of cold recovery between sets to help reverse the effects of fatigue which may lead to increase strength endurance gains; 2) pre-weight training to warm up muscles in the arms, back, shoulders or chest; 3) with hot/cold contrast recovery to recover after a weight training workout; 4) using cold recovery after a weight training workout if a user plans to workout the upper body on short rest; and 5) with blood flow restriction training as a means to increase the difficulty and adaptation response from a user's workouts. Meanwhile, some common uses with endurance training may comprise 1) pre-endurance workout to help warm up or pre-cool the body and muscles; 2) on rest intervals to cool the body; 3) post-workout to help kick start the cool down and recovery process; 4) increasing the difficulty of a user's workouts with heat sessions or heat training which lead to increased adaptation.

An alternative sporting use case may occur with baseball pitchers as either a cooling recovery tool or warm up tool. The negative pressure compression may act as a perceptual recovery technique that feels good when in use because it is tight. While the use of heat/cold may feel good to the touch. Meanwhile, physiologically the use of a heat/cold source applied to the palm may increase vasodilation which may then increase thermoregulation. Ideally, a pitcher could use the negative pressure compression sleeve with a heating source to warm the arm before starting to throw or warm the arm between innings. Between innings a pitcher could cool the arm via the use of palm cooling which may help reverse fatigue or increase endurance. After using a cold source, a pitcher may want to rewarm the arm 1-3 minutes in preparation of throwing again. While post pitching performance, a pitcher could use the negative pressure recovery sleeve with cold recovery to increase their rate of recovery by reducing fatigue, stress and inflammation while also adding the perceived recovery and perceived performance benefits.

FIG. 1 is an exterior view of an embodied device 100 designed for use with an arm extremity 114 designed for use before, during, and after a workout. In this case the apparatus 100 includes a thermal regulator in the form of a gel pack 112, a fatigue stimulator in the form of a restrictive cuff 110 and a negative pressure circulatory pull in the form of a vacuum apparatus including a suction valve 102, a suction squeeze pump 104 (which may also include a plug, not shown, to prevent air leakage at the end of the squeeze pump) and a vacuum sealed shrink wrap enclosure 106, that as air is pumped out of the space between the apparatus 100 and the users extremity 114 by pumping the suction squeeze pump which draws the air through the suction valve or aperture 102 into the pump 104 and is pumped out of the pump and into the ambient environment. This voiding of the surrounding air out of the apparatus 100 and into the ambient environment causes the vacuum sealed shrink wrap enclosure 106 to shrink and conform around the users hand, palm, and arm 114. The top of the device shows a hook and loop 108 fastening system with a material such as Velcro 110, that is partially sewed and glued to the vacuum sealed shrink wrap enclosure 106 which is needed to complete the seal and allow for the creation of a negative pressure environment. The restrictive cuff 110 is placed through the hook and loop 108 and can be pulled tightly to restrict the blood flow at the top of one's arm when practicing hypoxia training. The ideal restriction of blood flow is 50-70% at the top of the arm. If the underlying or inside material was made of nylon instead of rubber on the shrink wrapped sleeve 106, then a negative pressure would only be held briefly, 2-15 seconds before dissipating, which would require constant squeezing of the squeeze pump in order to hold a negative pressure environment. Although the present disclosure is shown with three physiological repair enhancers: heat exchange, hypoxia and negative pressure controls; alternative embodiments may include all of the potential combinations of physical recovery enhancers that are found in these drawings.

FIG. 2 is an exterior view of an embodied device 200 designed for use with an arm extremity 214 but without a negative pressure embodiment. In this case the apparatus 200 includes a thermal regulator in the form of a gel pack 212, a fatigue stimulator in the form of a hook and loop fastening system 208 and stretchable Velcro cuff 210, and a recovery aiding compression sleeve 206. The gel pack 212 rests inside a strategically placed pouch 216 to cover the palm of the hand and bottom of the arm 214. The compression sleeve 206 has a thumb opening 218 to give the thumb a free range of motion, and it also has a fingers opening 220 to give the fingers a free range of motion. When in use the user puts a cold gel pack 40-80 degrees Fahrenheit into the pouch 216 to cool the palm of the hand and the under arm and uses it during athletic activities or when the body is at rest between exercise sets or after exercise sets to enhance recovery.

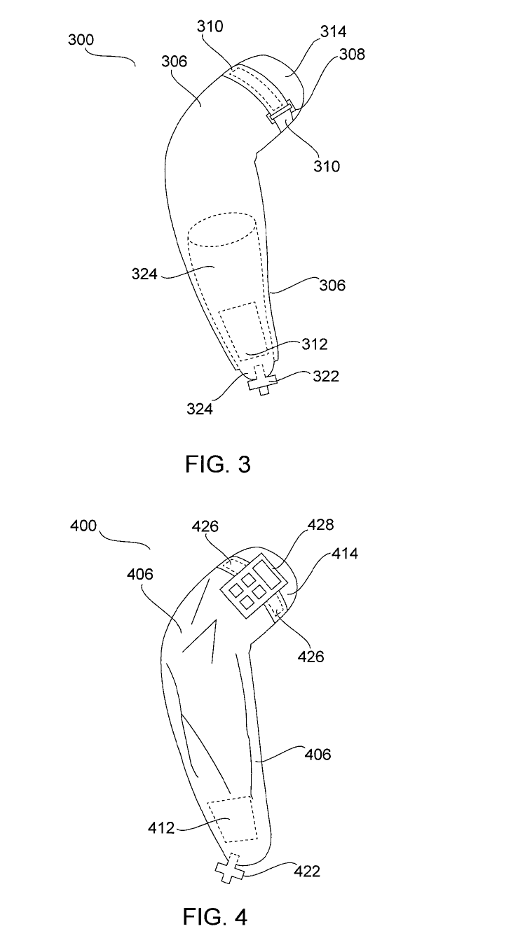

FIG. 3 is an exterior view of another embodied device 300 designed to control thermoregulation via the sleeve itself. The sleeve is a two part sleeve where the top sleeve 306 stays on during physical activity, while the bottom gel sleeve 324 can be removed when the hand needs to grip a weight bar, weight, or any other object. The underlying material of the bottom gel sleeve 324 is made of gel on one side and nylon on the other. The gel side is meant to contact and cool the arm appendage 314. Other combinations of cooling material may also be used to control the thermoregulation of an arm appendage 314 while in use with the goal of creating and holding a negative pressure environment through the use of an automatic vacuum generator 322. Any other cooling material or wicking fabric that wicks away sweat will provide an additional thermoregulation benefit. The bottom gel sleeve 324 allows for the refrigeration or cooling before using it. It can also be removed and refrigerated during an activity, so that it can be applied immediately after the activity or intermittently. Also, multiple bottom gel sleeves 324 can be stored and refrigerated or cooled, and then rotated in to replace a bottom gel sleeve 324 that is currently in use to help optimize the thermoregulation process. Cooling the bottom gel sleeve 324 will allow the arm to be cooled on the outside, while the palm holds a cold pack 312 between 40 and 80 degrees Fahrenheit, which cools the blood and then cools the muscles from inside the body to provide an inside outside technique to combat heat fatigue. At the top of the sleeve, a hook and loop fastening system 308 is used with a stretchable Velcro material 310, which is attached to the outside surface of the top sleeve 306, and aids in sealing the sleeve so that it can hold a negative pressure environment while also acting as a fatigue stimulator if fastened tight enough around the arm appendage 314. In order to create the negative pressure environment either the top sleeve 306 or bottom gel sleeve 324 can overlap with the other piece in order to hold the negative pressure environment, as both ways can create an airtight seal. Ideally the bottom gel sleeve 324 is put on first, and then the top sleeve 306 is put on over top of it. When replacing the bottom gel sleeve 324 with another refrigerated bottom gel sleeve 324, or when replacing the cold pack 312, the bottom gel sleeve 324 is to be pulled and should slide off the arm appendage 314. When the bottom gel sleeve 324 needs to be put back on when the top sleeve 306 is already on, then the top sleeve 306 can be rolled up to allow the bottom gel sleeve 324 to be fully applied before rolling back down and covering the bottom gel sleeve 324 with the top sleeve 306. Alternatively, the bottom gel sleeve 324 can be pulled over the top sleeve 306. The automatic vacuum generator 322 can be permanently or temporarily secured to the bottom gel sleeve 324. If the automatic vacuum generator 322 is temporarily sealed then this will allow for it to be removed and reapplied during rotational use of the bottom gel sleeve 324 after a refrigeration period.

FIG. 4 is an exterior view of another embodied device 400 designed to control thermoregulation and blood flow for use with an arm extremity 414. In this embodiment the apparatus 400 includes a thermal regulator in the form of a gel pack 412, a fatigue stimulator in the form of an inflatable cuff 426 and automatic pressure setting controller 428 and a negative pressure circulatory pull in the form of a an automatic vacuum generator apparatus 422, which is attached to a shrink wrapped sleeve 406 that conforms to the user's arm appendage 414. The inflatable cuff 426 is constructed through a rubber lining that wraps around the top of the arm and is connected to an automatic pressure setting controller 428 with the goal of restricting the blood flow by setting the pressure of an user based on arm circumference size and then applying that information to apply a pressure in the 50-70 percent of complete blood flow restriction, where when using a scale of 1-10 with 10 being complete blood flow restriction, then the automatic pressure setting controller would set to a 5-7, which allows for enough blood flow to travel through the arteries and down the arms to the hands to make it safe to use for healthy individuals. A pressure setting that restricts 50-70 percent of the blood flow is enough to restrict the surface veins and pool the blood below the cuff, which floods the muscles with blood and induces muscle cell swelling and metabolic build up. If the cuff were applied at top of the arm appendage 414, then that would cause muscle cell swelling in the triceps and biceps, which creates a permanent increase in muscle size in the triceps and biceps and that may occur even in the absence of weight training. While blood flow restriction training is typically applied at low intensity while lifting light weights, which is considered 10-30% of your one max rep, it can also be performed with high intensity and high loads. For many using blood flow restriction training with light weights and low intensities is uncomfortable or painful, but the pain or discomfort is less noticeable when using blood flow restriction on rest intervals and then performing high intensity and high load exercises. High intensity workouts can be used with blood flow restriction by restricting the blood flow of the non-weight training arm and then using unilateral circuits to perform multiple bicep and tricep exercises. Because only the blood flow on the rest interval of the arm not in use is restricted, the free, non-restricted arm may lift heavier weight that can range in the 8-12 rep to failure range. Using heavier weight may help result in an increase in muscle hypertrophy compared to using light weights in the 10-30% of one rep max. When you combine and use the muscle cell swelling fatigue stimulator on an arm that is in a state of rest while performing a high intensity unilateral arm circuit where weight is lifted in the 8-12 rep to failure range, an increase in muscle growth may occur over a short period of time. Ideally the resting arm and the arm performing the unilateral circuit are to be rotated throughout the workout, so that both arms experience the same workout conditions and perform the same amount of sets. While increased muscle swelling occurs through the use of an inflatable cuff 426 and automatic pressure setting controller 428, the thermal regulator in the form of a gel pack 412, and the negative pressure circulatory pull in the form of a an automatic vacuum generator apparatus 422 also play a role in the process. When the thermal regulator in the form of a gel pack 412 is used between 40-80 degrees Fahrenheit, it helps to cool the blood, which may slow the flow of the blood back up the arm, helping the blood to pool in the muscles. When the gel pack 412 is used between 40-80 degrees Fahrenheit, it may also help to offset some of the fatigue brought forth by the fatigue stimulator, which may lead to increased recovery from DOMS and in muscle function in future days. Lastly the automatic vacuum generator apparatus 422 pulls the blood in the palms of the hand towards the surface, which also slows the flow of blood out of the hands, and increases the time in contact between the surface blood and the thermal regulator in a form of a gel pack 412. Additionally, the thermal regulator in a form of a gel pack 412 when used with a shrink wrapped sleeve 406 before, during, or after high intensity unilateral circuits or exercises where heavier weights are lifted in the 3-12 rep to failure range to help increase endurance and muscle activation and allow for more reps to be completed while simultaneously allowing for active recovery by helping to combat heat fatigue, inflammation, and stress. This may allow for the user to increase frequency/volume without increasing their recovery time, which when used with a fatigue stimulator in the form of an inflatable cuff 426 and automatic pressure setting controller 428 allows for an increased use of and results from applying blood flow restriction training.

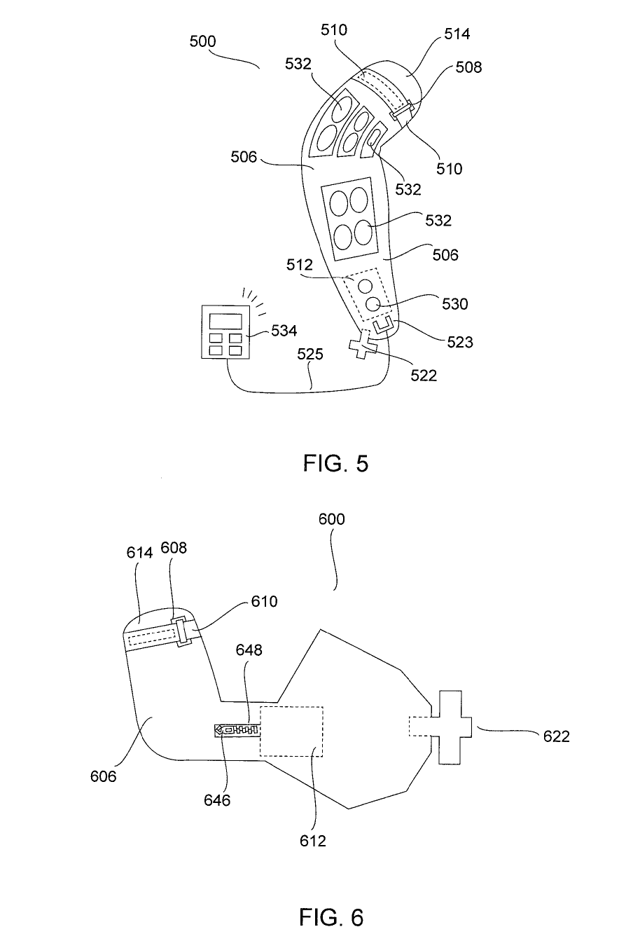

FIG. 5 is an exterior view of the embodied device 500 designed to control thermoregulation and blood flow for use with an arm extremity 514 while collecting and displaying relevant data about the user's body and workout recovery data. In this case the apparatus 500 includes a thermal regulator in the form of a gel pack 512, a fatigue stimulator in the form a hook and loop fastening system 508 and stretchable Velcro cuff 510, and a negative pressure circulatory pull in the form of a an automatic vacuum generator apparatus 522, which is attached to a shrink wrapped sleeve 506 that conforms to the user's arm appendage 514. Temperature sensors 530 are placed inside the thermal regulator in the form of a gel pack 512, and could also be placed inside the sleeve near the palm to accurately gauge the temperature of the gel pack 512. Body sensors 532 are fastened to the inside of the shrink wrapped sleeve 506 and are strategically placed along the arm and on the biceps and triceps. The information collected from the temperature sensors 530 and the body sensors 532 is transmitted and collected by the hand held data receiver 534, which then displays the body and workout recovery information to the end user, such as localized information about the muscles (temperature, and other general body or workout recovery information like lactate levels, lactate clearance, blood oxygen saturation percentages, cortisol, creatine kinase and dehydration testing to name some of the possible recovery data that could be collected, transferred, interpreted or displayed to the end user. The hand held data receiver 534 may be in the form of a cell phone, and the data may be accessed through a programmed application that gathers the data from the sensors inside the sleeve so that it can display the user's information to themselves. While a heart rate variability finger sensor and monitor 523 may be connected to a finger appendage and transmitted to a hand held data receiver 534 via a transmitter cord 525 or wirelessly via Bluetooth or another wireless-like transmitting device or application. The finger sensor 523 uses infrared light that emits light from one end of the finger sensor to the other end of the finger sensor where the sensor measures the changes in the amount of light received as blood is constantly pumped around the body and the blood density changes in the finger with every heart beat. This information is collected by the finger sensor and may be transmitted via a transfer cord 525 to a user's phone that is calibrated via an algorithm to provide the user with reliable heart rate variability and heart rate measurement data for the end user to assess their recovery from exercise. While a transfer cord 525 is used, it should also be understood that data may be transmitted wirelessly.

FIG. 6 is an exterior view of an alternative embodied device 600 designed to control thermoregulation for use with the hand, wrist, and arm extremity 614. In this case the apparatus 600 includes a thermal regulator in the form of a gel pack 612, a negative pressure circulatory pull in the form of an automatic vacuum generator apparatus 622, which is attached to a vacuum sealed sleeve 606 that conforms to the user's arm and hand appendage 614 with the help of a restrictive cuff 610 that completes the seal on the end of the vacuum sealed sleeve 606. A fatigue stimulator is also present in the form a hook and loop fastening system 608 and a restrictive cuff 610. The palm and hand extremity 614 hold the thermal regulator in the form of a gel pack 612 during use. The embodied device 600, also includes a zipper 646 and zipper tracks 648 in the wrist area of the vacuum sealed sleeve 606 to allow for the removal of the cold pack 612 and to slip the hand extremity 614 in and out of the vacuum sealed sleeve 606, which allows the embodied device 600 to be used during a workout where the palm or hands need to grip a dumbbell, weight bar, or perform an athletic activity. It can also be used to practice blood flow restriction training using light weights with out removing the vacuum sealed sleeve 606, as the hand extremity 614 could slip out to perform weight training. The use of a compression like vacuum sealed sleeve 606 when performing blood flow restriction training is an improvement over traditional blood blow restriction training where only a cuff is used at the top of the arm. The tight or shrink wrapped sleeve applies firm pressure to the superficial veins beneath the sleeve. This firm pressure helps control the size of the superficial veins and prevents them from over expanding with blood, which in turn prevents the pooling of the blood in those superficial veins. The firm pressure helps the blood flow more easily up the arm extremity 614, which may also help prevent blood clotting and make blood flow restriction training safer.

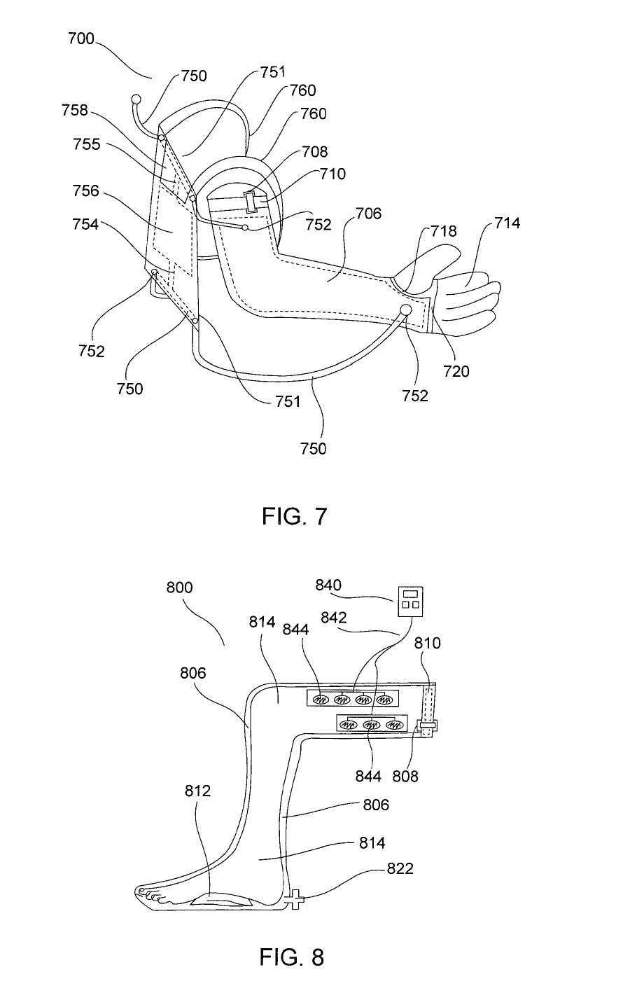

FIG. 7, the apparatus 700 includes a thermal regulator in the form of a circulating fluid backpack apparatus 751, comprises a fluid chamber 756 that pumps fluid into outlet connection valve 755 that connects the fluid chamber 756 to the transfer tubes 750. The transfer tubes 750 at the top of the circulating fluid backpack apparatus 751 connect to the gasket 752, which connects with the fluid transfer sleeve 706. The fluid enters the fluid transfer sleeve 706 at the gasket 752 which is found at the top of the arm, and is below the fatigue stimulator in the form a hook and loop fastening system 708 and a restrictive cuff 710. The transferred fluid runs down the middle of the fluid transfer sleeve 706 in between the outer and inner parts of the rubber fluid transfer sleeve 706 before existing near the wrist area, where the gasket 752 interacts with the transfer tubes 750. This allows the hand and arm appendage 714 to be cooled during exercise. After the fluid leaves the bottom part of the fluid transfer sleeve 706, it travels back to the fluid chamber 756 by way of the transfer tubes 750, which flow into the input connection valve 754 before entering the fluid chamber 756, which can be heated or cooled. The inside of the circulating fluid backpack apparatus 751 can be accessed via the backpack cover 758. The fluid transfer sleeve 706 has a thumb opening 718 to give the thumb a free range of motion, and it also has a fingers opening 720 to give the fingers a free range of motion. The circular cooling apparatus 700 is secured to the shoulders and back of an user through backpack straps 760.

FIG. 8 is an interior view of an embodied device 800 designed for use with the foot, and the upper and lower leg 814. In this case the apparatus 800 includes a thermal regulator in the form of a gel pack 812, a negative pressure circulatory pull in the form of an automatic vacuum generator apparatus 822 and a vacuum sealed shrink wrap enclosure 806, that as air is pumped out of the space between the apparatus 800 and the users extremity 814 by the automatic vacuum generator apparatus, air is removed from inside the shrink wrap enclosure 808 and is pumped out into the ambient environment. This voiding of the surrounding air out of the apparatus 800 and into the ambient environment causes the vacuum sealed shrink wrap enclosure 806 to shrink and conform around the user's foot, and the upper and lower leg 814. Additionally, in this apparatus 800 there is a thermoregulation enhancer in the form of a gel-pack 812 placed on the underside of the foot. A fatigue stimulator is also present in the form a hook and loop fastening system 808 and stretchable Velcro cuff 810. While a muscle release facilitator in the form of a electric stimulation apparatus including electric stimulation pads 844 which are secured strategically on the area to be treated of the user, the electric stimulation apparatus further includes electric stimulation leads 842 which communicate the electric stimulation pads 844 with the electric stimulation control box 840, additionally the negative pressure sleeve cover 806 may include small sealable apertures or sealed e-stimulation lead tie-ins (not shown) which facilitates the communication of the leads 842 from the box 840 and into the apparatus 800 and to the stimulation pads 844.

FIG. 9 is an exterior view of an embodied device 900 designed to control thermoregulation and increase blood flow for use with an arm extremity 914. In this case the apparatus 900 includes a thermal regulator in the form of a gel pack 912, a negative pressure circulatory pull in the form of an automatic vacuum generator apparatus 922 and a vacuum sealed shrink wrap sleeve 906. The embodied device 900 increases blood flow through a viscosity enhancement feature in the form of magnets 962 attached strategically to the hand area of the inside of the vacuum sealed shrink wrap sleeve 906 and also via a point pressing pressure system in the form of plastic disk 964 with sharp points 966 that are attached to the inside of the vacuum sealed shrink wrap sleeve 906. The point pressing system in the form of plastic disks 964 with sharp points 966 can be used prior to a workout to warm up and loosen the muscles that will be worked out such as the bicep and tricep muscles, or used after a workout to kick start the recovery process by flushing blood into the targeted area, as the vacuum sealed shrink wrap sleeve 906 pushes the point pressing system onto the arm extremity 914 and applies pressure when air is removed from inside the sleeve. The thermal regulator in the form of a gel pack 912 may be heated before a workout to warm up the muscles or after a workout to stimulate blood flow in a targeted area and aid in the overall recovery process. In order to create a vacuum sealed shrink wrap sleeve 906, the seams of the sleeve can be sealed with a silicone or rubber adhesive to help sustain a negative pressure environment. If the underlying material is made of nylon, it will only hold a negative pressure environment briefly, roughly 2-15 seconds, which means the vacuum would need to be continuous in order to sustain a negative pressure environment.

Although the invention has been described with reference to the above example, it will be understood that modifications and variations are encompassed within the spirit and scope of the invention. Accordingly, the invention is limited only by the following claims.

What is claimed is:

1. An apparatus for the enhanced recovery from injury or body fatigue, stress or inflammation due to strenuous activity or strenuous conditions of a subject comprising:
a combination of two or more recovery enhancers selected from;
a) a thermoregulation enhancer comprising a cooling or a heating source or a temperature cooling wicking fabric;
b) blood flow viscosity enhancer comprising an electromagnetic source, a compression source; or an electric stimulation source;
c) a circulatory pull comprising a negative pressure or vacuum source capable of drawing more blood to a treatment area or capable of increasing vasodilation; and
d) a muscle release apparatus comprising one or more pressure point applicators or electric stimulation devices.

2. The apparatus of claim 1, wherein the heating source heats above 98.6 degrees Fahrenheit and the cooling source cools below 98.6 degrees Fahrenheit.

3. The apparatus of claim 1, wherein the thermoregulation enhancer comprising the cooling source, the heating source or the temperature cooling wicking fabric; further comprise of a gel, liquid or chemical agent form.

4. The apparatus of claim 1, wherein heat transfer exchange is enhanced using a vasodilation source.

5. The apparatus of claim 1, wherein said apparatus further comprises:
a negative pressure shrink wrap enclosure or compression enclosure that squeezes an appendage of a user.

6. The apparatus of claim 5 wherein the enclosure comprises a negative pressure greater than 0 mm Hg relative to atmospheric pressure.

7. The apparatus of claim 5 wherein the enclosure covers an arm, leg or hand appendage of a user.

8. The apparatus of claim 5 wherein the enclosure comprises a negative pressure environment with a heat exchange surface, a pressure point application, a blood flow modifier, and an adaptation enhancer or a recovery tracking apparatus.

9. The apparatus of claim 5 comprising a thermoregulation enhancer within the underlying material of the enclosure.

10. The apparatus of claim 5 wherein said negative pressure enclosure further comprises a manual or automatic vacuum generator.

11. The apparatus of claim 5 wherein said negative pressure comprises rubber, neoprene or an impenetrable material that prevents air-loss.

12. The apparatus of claim 5 wherein said negative pressure comprises a impenetrable sealant or a cuff to seal the negative pressure environment.

13. The apparatus of claim 8 comprising a recovery tracking system capable of collection, interpretation, transferring or displaying of recovery data.

14. The apparatus of claim 13 wherein the recovery tracking system comprises measurements that provide an end user with recovery data, information, statistics or recommendations.

15. The apparatus of claim 13 wherein the recovery tracking system comprises interpretation of data customized for an individual end user.

16. The apparatus of claim 13 wherein the recovery tracking system comprises heart rate, heart rate variability, lactate levels, lactate clearance, blood oxygen saturation percentages, cortisol, creatine kinase or dehydration monitoring.

17. An apparatus for improving blood flow comprising:
a magnetic field source,
a heating source,
an electric stimulation source, and
a negative pressure shrink wrap enclosure or compression enclosure that squeezes an appendage of a user.

18. An apparatus for increasing vasodilation comprising:
a negative pressure environment source;
heat transfer exchange mechanism; and
a blood flow restrictive cuff to increase carbon dioxide.
